(19) United States
(12) Patent Application Publication
(10) Pub. No.: US 2014/0150806 A1
(43) Pub. Date: Jun. 5, 2014

(54) Robotic First Responder System and Method

(71) Applicants: John Hu, Boxborough, MA (US); Yi-Je Lim, Boxborough, MA (US)

(72) Inventors: John Hu, Boxborough, MA (US); Yi-Je Lim, Boxborough, MA (US)

(21) Appl. No.: 13/691,840

(22) Filed: Dec. 2, 2012

(51) Int. Cl. A61B 19/00; A61B 5/00; A61G 1/003; A61F 5/37; A61G 1/02

(52) U.S. Cl. CPC A61B 19/2203; A61F 5/3769; A61G 1/0275; A61G 1/003; A61B 5/6887; A61B 5/7465; Y10S 901/01; Y10S 901/46
USPC 128/870; 901/1; 901/46

(57) ABSTRACT

A robotic first responder system provides injured patients with first response medical care and extracts the injured patient from a dangerous or remote area. After assessing of the level of consciousness of an injured patient, the robotic unit utilizes an inflatable immobilization device for safe robotic rescue of injured patients. The robotic unit lifts and transfers an injured patient to an extraction vehicle, a secure, bullet-proof wheeled system that can be pulled by the robot to safe location. The extraction vehicle continues serving as the evacuation stretcher to a medical station. The robotic unit utilizes zero moment point control to maintain stability while lifting the patient. A robot first responder tows the extraction vehicle or the extraction vehicle self-navigates incorporating with power drive and autonomous navigation systems.

### FEDERALLY SPONSORED RESEARCH OR DEVELOPMENT

This patent is partially developed through US Army STTR Phase II Project under Contract W81XWH-11-C-0007 with project title: A Near Autonomous Combat Casualty Extraction Robotic System (called c2Exbot Project), and through US Army STTR Phase II Project under Contract W81XWH-08-C-0116 with project title: Robotic Noninvasive Neck & Spinal Injury Assessment Device (namely cRoNA—Combat Robotic Nursing Assistant).

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

One or more embodiments of the invention generally relate to robots. More particularly, one or more embodiments of the invention relate to first responder robots, namely combat casualty evacuation and casualty care robots.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that a robot is a mechanical or virtual intelligent agent that can perform tasks automatically or with guidance, typically by remote control. In practice a robot is usually an electro-mechanical machine that is guided by computer and electronic programming. By mimicking a lifelike appearance or automating movements, a robot may convey a sense that it has intent or agency of its own.

Typically, a first responder is a person who has completed a course and received certification in providing pre-hospital care for medical emergencies. The first responder may have more skill than someone who is trained in basic first aid but is typically not a substitute for advanced medical care rendered by emergency medical technicians, emergency physicians, nurses, or paramedics.

Typically, the use of stabilization techniques improves the chances of a person surviving the transport to the nearest trauma-equipped hospital. After ensuring their own safety and taking isolation precautions, a primary survey is performed, consisting of checking and treating airway, breathing, and circulation, then an assessment of the level of consciousness. To prevent further injury, unnecessary movement of the spine is minimized by securing the neck with a cervical collar, and the back with a long spine board with head supports, or other medical transport device such as a Kendrick extrication device, before moving the person.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates a detailed perspective view of an exemplary robotic first responder positioned to lift a casualty, in accordance with an embodiment of the present invention;

FIG. 2 is a flow chart illustrating an exemplary process for steps utilized by a robotic first responder system and method, in accordance with an embodiment of the present invention;

FIGS. 3A, 3B, and 3C illustrate detailed perspective views of the robot first responder enacting the steps of the robotic first responder system and method, in accordance with an embodiment of the present invention;

FIGS. 4A, 4B, and 4C illustrate detailed perspective views of an exemplary robotic first responder positioning an immobilization device around the patient/casualty body, in accordance with an embodiment of the present invention;

FIGS. 5A, 5B, and 5C illustrate detailed perspective views of an exemplary stabilizing device, in accordance with an embodiment of the present invention;

FIGS. 6A and 6B illustrate detailed perspective views of an exemplary extraction vehicle, in accordance with an embodiment of the present invention, where FIG. 6A illustrates an exemplary extraction vehicle with the lid closed, and FIG. 6B illustrates an exemplary extraction vehicle with the lid open;

FIGS. 7A, 7B, and 7C illustrate views of an exemplary extraction vehicle in various positions, in accordance with an embodiment of the present invention;

FIGS. 8A, 8B, and 8C illustrate detailed perspective views of an exemplary sequential image of a pair of wheels with a unique 4-wheel methodology, providing simultaneous support for bump absorption and heading control wheel on the axle assembly in three varying positions, in accordance with an embodiment of the present invention;

FIGS. 9A and 9B illustrate detailed perspective views of an exemplary robot first responder towing the extraction vehicle, in accordance with an embodiment of the present invention, where FIG. 9A illustrates an exemplary scenario of the extraction vehicle being towed by the robot first responder, and FIG. 9B illustrates an exemplary scenario of the extraction vehicle incorporating power drive and autonomous navigation systems, which enable multiple extraction vehicles to self-navigate to safety after receiving the injured patient;

FIGS. 10A, 10B, and 10C illustrate detailed perspective views of an exemplary scenario of the robot first responder pulling the patient to safety in a combat zone, in accordance with an embodiment of the present invention; and,

FIG. 11 illustrates a typical computer system that, when appropriately configured or designed, can serve as a computer system in which the present invention may be embodied.

Unless otherwise indicated illustrations in the figures are not necessarily drawn to scale.

### DETAILED DESCRIPTION OF SOME EMBODIMENTS

Embodiments of the present invention are best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

In the following description and claims, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

“Software” may refer to prescribed rules to operate a computer. Examples of software may include: code segments in one or more computer-readable languages; graphical and or/textual instructions; applets; pre-compiled code; interpreted code; compiled code; and computer programs.

A “computer-readable medium” may refer to any storage device used for storing data accessible by a computer. Examples of a computer-readable medium may include: a magnetic hard disk; a floppy disk; an optical disk, such as a CD-ROM and a DVD; a magnetic tape; a flash memory; a memory chip; and/or other types of media that can store machine-readable instructions thereon.

A “computer system” may refer to a system having one or more computers, where each computer may include a computer-readable medium embodying software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those made through telephone or other communication links. A network may further include hard-wired connections (e.g., coaxial cable, twisted pair, optical fiber, waveguides, etc.) and/or wireless connections (e.g., radio frequency waveforms, free-space optical waveforms, acoustic waveforms, etc.). Examples of a network may include: an internet, such as the Internet; an intranet; a local area network (LAN); a wide area network (WAN); and a combination of networks, such as an internet and an intranet.

Exemplary networks may operate with any of a number of protocols, such as Internet protocol (IP), asynchronous transfer mode (ATM), and/or synchronous optical network (SONET), user datagram protocol (UDP), IEEE 802.x, etc.

Embodiments of the present invention may include apparatuses for performing the operations disclosed herein. An apparatus may be specially constructed for the desired purposes, or it may comprise a general-purpose device selectively activated or reconfigured by a program stored in the device.

Embodiments of the invention may also be implemented in one or a combination of hardware, firmware, and software. They may be implemented as instructions stored on a machine-readable medium, which may be read and executed by a computing platform to perform the operations described herein.

In the following description and claims, the terms “computer program medium” and “computer readable medium” may be used to generally refer to media such as, but not limited to, removable storage drives, a hard disk installed in hard disk drive, and the like. These computer program products may provide software to a computer system. Embodiments of the invention may be directed to such computer program products.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In a similar manner, the term “processor” may refer to any device or portion of a device that processes electronic data from registers and/or memory to transform that electronic data into other electronic data that may be stored in registers and/or memory. A “computing platform” may comprise one or more processors.

A non-transitory computer readable medium includes, but is not limited to, a hard drive, compact disc, flash memory, volatile memory, random access memory, magnetic memory, optical memory, semiconductor based memory, phase change memory, optical memory, periodically refreshed memory, and the like; however, the non-transitory computer readable medium does not include a pure transitory signal per se; i.e., where the medium itself is transitory.

The present invention will now be described in detail with reference to embodiments thereof as illustrated in the accompanying drawings.

There are various types of robotic first response systems and methods 100 that may be provided by preferred embodiments of the present invention. For example, without limitation, the robotic first response system and method may provide a robotic first responder 102 that provides an injured patient with first response medical care, and extracts the injured patient 104 from a dangerous or remote area. Those skilled in the art, in light of the present teachings, will recognize that the robotic first responder may include a humanoid robot. In some embodiments, the robotic first responder may have numerous components efficacious for extracting the injured patient from a combat zone, including, without limitation, a holonomic drive system for easy maneuverability in a combat zone, an intuitive interface for human-robot interaction, and at least one bimanual dexterous manipulator having sufficient strength to lift and move the patient. In some embodiments, the robot first responder may include numerous components that are integrated together. Some of the major components may include, without limitation, at least one bimanual dexterous manipulator, an innovative humanoid upper torso, a drive track with a stability enhancement device, a navigation control system with 3D sensing and perception capability, and an interface for human-robot interaction.

In one alternative embodiment, the robotic first responder performs numerous functions that facilitate the first response needs of soldiers in a combat zone, including, without limitation, receiving commands from a medical professional to service the patient's medical needs, reducing the medical professional's exposure to back pain from heavy lifting, entertaining the patient, moving heavy objects, carrying medical supplies, acting as a conduit for remote medical professionals, and protecting the soldier/patient from projectiles. In yet another alternative embodiment, the medical robotic system may provide numerous capabilities efficacious for servicing patients in a battle zone medical facility, including without limitation: a) navigating intelligently in medical facility with a mobile drive track, b) supporting an intuitive interface guided by human-robot interaction, c) performing through direct control by a health professional through a telepresence operation, and d) providing dexterous manipulation and sufficient strength to lift/move patients and heavy loads.

In yet another alternative embodiment, the robotic first responder may approach the patient on the battle zone. The robotic first responder may adjust the elevation of the torso so that the interface is at eye level with the patient. A drive train may raise or lower to adjust the height of the torso. In one embodiment, a medical professional including, but not limited to, a military doctor, nurse, and technician may communicate through visual and audio means to the patient. In one alternative embodiment, the medical professional may be remotely located from the battle zone. The patient may also write instructions and questions on a touch screen positioned on the interface. In some embodiments, the medical professional may transmit instructions to robotic first responder to approach and lift the patient. The robotic first responder may increase the base with a stability enhancement device on the drive train to ensure secure control of patient. A pair of dexterous manipulators may lift the patient with sufficient torque so that up to, but not limited to three hundred pounds may be lifted. In some embodiments, the dexterous manipulators may comprise of a fabric efficacious for providing sensitive contact with human skin.

In some embodiments, the injured patient may be immobilized prior to be rescued from a combat zone under a hazardous battlefield scenario. The robot first responder may be controlled by a remote operator through various modes, including, without limitation, telepresence mode, supervisory mode and semi-autonomous mode. In some embodiments, remote control may be utilized to control the robot first responder, and a camera may allow the remote operator to have a telepresence at the scene. Those skilled in the art, in light of the present teachings, will recognize that the remote operator and/or the robot first responder must initially assess 200 the injured patient by localizing the patient and determining the optimal position for the patient. In the assessment, a primary survey may be performed through the robot first responder, including, without limitation, observing the level of consciousness of the patient, communicating with the patient, checking the position of the body, checking the airway, checking for breathing, and checking for circulation. In some embodiments, the robot first responder may then prepare 210 the patient for transport by positioning the patient into a prone position. In some embodiments, the prone position may include, without limitation, a position with the patient lying face down with arms bent comfortably at the elbow. To prevent further injury, unnecessary movement of the spine is minimized by securing the neck and the back with an immobilization device 120. The immobilization device may be efficacious for transporting patients who may suffer from a spine or other serious injury requiring immobilization for safe movement. In particular, injuries that the robot first responder may recognize as requiring stabilization of the patient may include, without limitation, spinal injuries, traumatic head injuries, and copious amounts of bleeding. The robotic first responder may then position the patient onto the immobilization device for moving the patient with minimal stress and movement to the neck and spine. The stabilizing device may inflate to conform around the patient's neck and spine.

In some embodiments, the robotic first responder may then lift and carry 220 the patient to an extraction vehicle 130. In some embodiments, the lift and carry method may include executing a wedge lift on the patient, where the arms may be compliantly wedged beneath the buttocks and shoulders. In some embodiments, the robotic first responder may utilize zero moment point control to maintain stability while lifting the patient. The patient may then be carried by the robot first responder to the extraction vehicle. The robot first responder may then lower the patient in the prone position onto a base platform in the extraction vehicle.

In some embodiments, the extraction vehicle may include a motorized medical stretcher designed for numerous medical functions, including, without limitation, protecting wounded patients in hazardous battlefield scenarios, monitoring vital signals of the patient, and effectively extracting patients from a combat hot zone. In some embodiments, the extraction vehicle may include a base platform 132 for receiving the patient, a lifting device 134 for assisting the robot first responder to lift the patient, and a lid portion 136 for covering and protecting the patient from battlefield hazards such as debris and bullets. Those skilled in the art, in light of the present teachings, will recognize that the axle assembly may utilize an Ackerman style steering system for maneuvering through tight areas and rough terrain. The axle assembly may ride on numerous assemblies, including, without limitation, four wheels, tracks, and sleds. However, in other embodiments, a normal rack-and-pinion steering may be utilized. In some embodiments, a navigation sensory portion 150 may guide the extraction vehicle away from dangers and obstacles in the combat zone. In some embodiments, a vital signs recording device 160 provides vital signs data capture and continuous patient monitoring, for bringing instant wireless vital signs communication between an ambulance and a medical facility. In some embodiments, a transmitter/receiver device 166 provides two-way communication between the patient and the remote operator. Those skilled in the art, in light of the present teachings, will recognize that the transmitter/receiver device may speed the time to provide effective care for time critical health events, including, without limitation, paralysis, brain trauma, bullet wounds, and heart failure. In one alternative embodiment, the robot first responder and the extraction vehicle may utilize a helicopter to arrive at the combat zone where the patient is located.

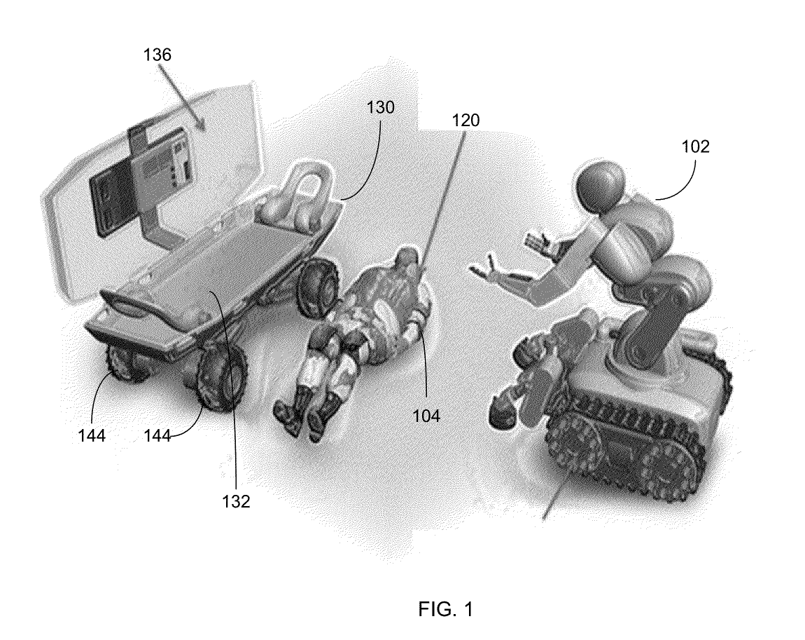

FIG. 1 illustrates a detailed perspective view of an exemplary robot first responder positioned to lift an injured patient, in accordance with an embodiment of the present invention. In some embodiments, the robot first responder may approach the injured patient in a combat zone. The robot first responder may adjust the elevation of the torso so that the interface is at eye level with the patient. A drive train may raise or lower to adjust the height of the torso. In one embodiment, a remote operator including, but not limited to, a soldier, doctor, nurse, and technician may communicate through visual and audio means to the patient. In one alternative embodiment, the patient may also write instructions and questions on a touch screen positioned on the robot first responder interface. In some embodiments, the remote operator may transmit instructions to the robot first responder to approach and transport the patient. The robot first responder may increase the stability base with a stability enhancement device on the drive train to ensure secure control of the patient. A pair of dexterous manipulators may position the patient onto the immobilization device, and lift the patient with sufficient torque. In some embodiments, the dexterous manipulators may comprise of a fabric efficacious for providing sensitive contact with human skin. Those skilled in the art, in light of the present teachings, will recognize that the dexterous manipulators may need to gently position a paralyzed patient on the immobilization device, and gently lift and carry the patient into the extraction vehicle. In some embodiments, the dexterous manipulator may comprise of series elastic actuators to better mimic the manipulation capabilities of human arms for providing an enhanced compliant manipulation.

In one alternative embodiment, the robot first responder may assists a soldier in battle by discharging a weapon, checking for mines, and acting as a bullet proof shield. The robot first responder may also assist a medical professional with servicing the patient's medical needs by reducing the medical professional's exposure to back pain from heavy lifting, entertaining the patient, moving heavy objects, carrying medical supplies, acting as a conduit for remote medical professionals. The robot first responder may also provide numerous capabilities efficacious for servicing patients in a medical facility, including but not limited to: a) navigating intelligently in hospital environments, b) supporting an intuitive interface guided by human-robot interaction, c) performing through direct control by a health professional through a telepresence operation, and d) providing dexterous manipulation and sufficient strength to lift/move patients and heavy loads. In alternative embodiment, the robot first responder may perform functions in a medical facility store house, such as, but not limited to, stocking medication, discarding waste, and taking inventory.

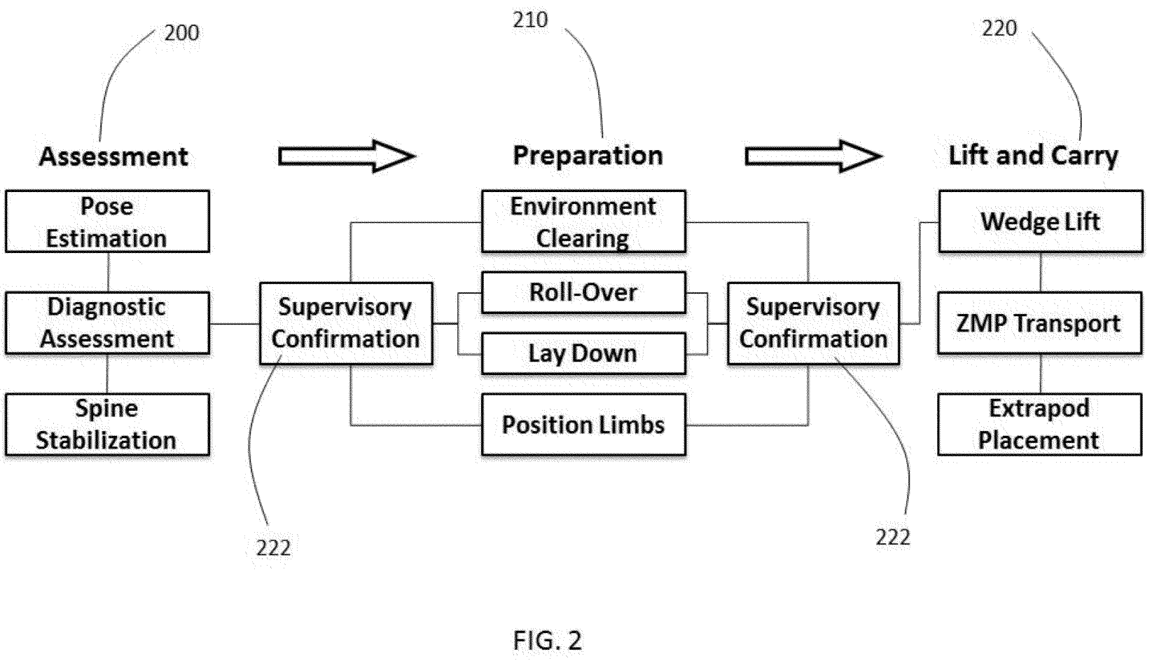

FIG. 2 is a flow chart illustrating an exemplary process for steps utilized by the robotic first responder system and method, in accordance with an embodiment of the present invention. The robotic first response systems and methods may include a control system for telepresence and semi-autonomous lifting and extraction of the injured patient. In some embodiments, the control system may allow for robust task execution as multiple objectives may run simultaneously within a prioritized scheduler. In some embodiments, the robot first responder's tasks may be divided three behavior phases, including, without limitation, assessment of the injured patient and the battlefield, preparation of the injured patient for movement and extraction, and lifting and carrying the patient to the extraction vehicle. In some embodiments, the remote operator may provide a supervisory conformation 222 between each phase of the operation. Those skilled in the art, in light of the present teachings, will recognize that since the robot first responder does not possess human intelligence, and the situation on the battlefield may evolve quickly, the remote operator must observe and analyze every action taken by the robot first responder. For example, without limitation, if the robot first responder is positioning the patient on the immobilization device, and debris begins to propel towards the patient, the remote operator may command the robot first responder to lie over the patient to provide cover against the debris. After the debris terminates, the robot first responder may continue positioning the patient onto the immobilization device. In some embodiments, the remote operator and/or the robot first responder must initially assess 200 the injured patient by localizing the patient and determining the optimal position for the patient. In an assessment, a primary survey may be performed through the robot first responder, including, without limitation, observing the level of consciousness of the patient, communicating with the patient, checking the position of the body, checking the airway, checking for breathing, and checking for circulation. In some embodiments, the robot first responder may then prepare 210 the patient for transport by positioning the patient into a prone position. In some embodiments, the prone position may include, without limitation, a position with the patient lying face down with arms bent comfortably at the elbow. To prevent further injury, unnecessary movement of the spine is minimized by securing the neck and the back with a immobilization device 120. In some embodiments, the robotic first responder may then lift and carry 220 the patient to an extraction vehicle 130. In some embodiments, the lift and carry method may include executing a wedge lift on the patient, where the arms may be compliantly wedged beneath the buttocks and shoulders. The robotic first responder may then utilize impedance control, to lift the patient into a position where the center-of-mass of the patient is between and below the elbows. This control algorithm may ensure that the patient does not slip from the robot first responder. In some embodiments, the first responder may utilize zero moment point control to maintain stability while lifting the patient. The patient may then be transferred by the robot first responder to the extraction vehicle. The robot first responder may then lower the patient in the prone position onto a base platform 132 in the extraction vehicle. The robot first responder may then close a lid portion 136 on the extraction vehicle and tow the patient to a desired location.

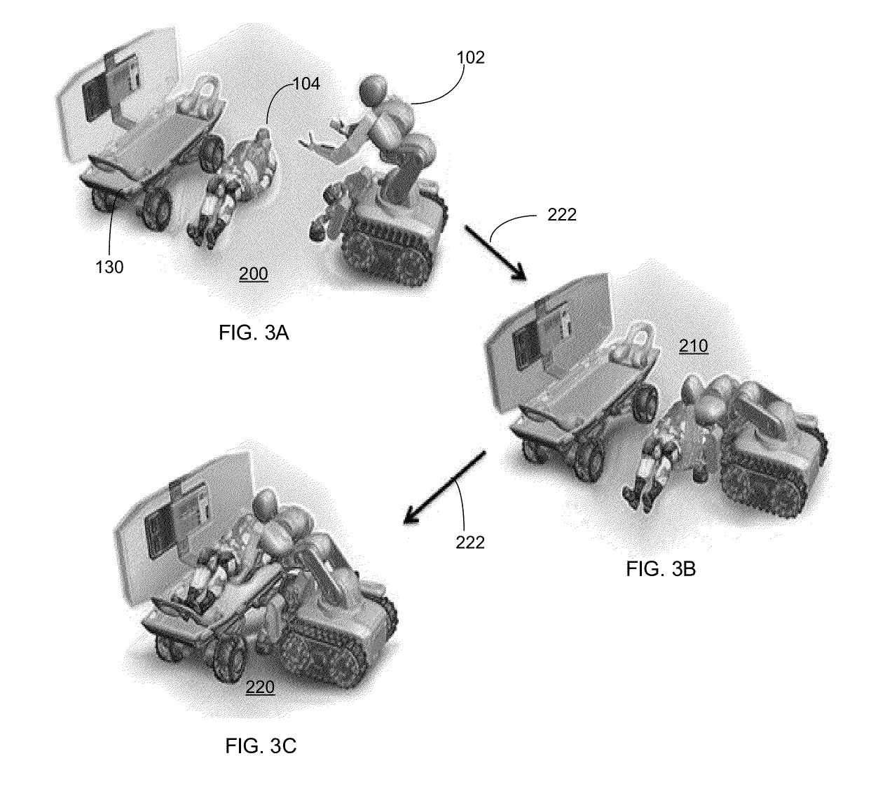

FIGS. 3A, 3B, and 3C illustrate detailed perspective views of an exemplary robotic first responder system and method with the robot first responder enacting the steps 222 of the robotic first responder system and method, in accordance with an embodiment of the present invention. In some embodiments, the robot first responder's tasks may be divided multiple behavior phases, including, without limitation, assessment of the injured patient and the battlefield 200, preparation of the injured patient for movement and extraction 210, and lifting and carrying the patient to the extraction vehicle 220.

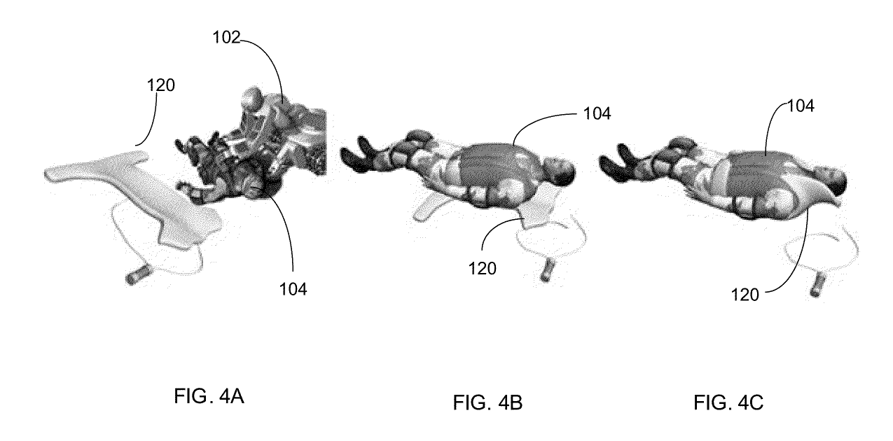

FIGS. 4A, 4B, and 4C illustrate detailed perspective views of an exemplary robot first responder positioning an immobilization device 120 around the patient body, in accordance with an embodiment of the present invention. In some embodiments, the immobilization device may function as a flexible body splint. In some embodiments, the immobilization device may require no straps and clasps that require difficult dexterous manipulation. FIG. 4A illustrates an exemplary immobilization device that may be a preformed bladder 120 that is laid on the ground like a blanket. FIG. 4B illustrates an exemplary patient 104 pulled onto the immobilization device. In some embodiments, the robot first responder may then inflate the immobilization device. In some embodiments, the immobilization device may automatically fill with fast-hardening foam materials. However, in other embodiments, other fluids may fill the stabilizing device, including, without limitation, air, water, and foam. FIG. 4C illustrates an exemplary immobilization device that inflates and automatically unfolds, conforming to the patient's head and spine. The fast-hardening foam material may quickly harden and the patient may be ready to be lifted by the robot first responder.

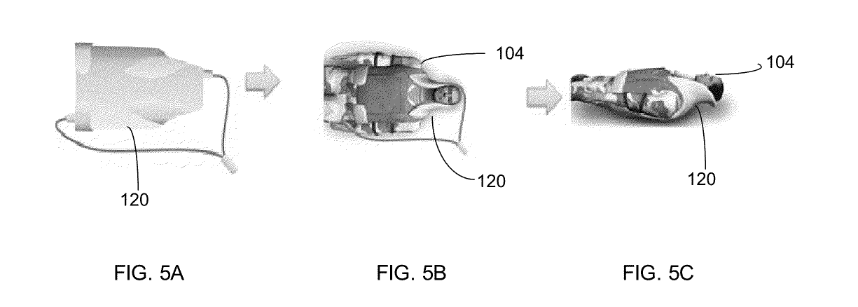

FIGS. 5A, 5B, and 5C illustrate detailed perspective views of an exemplary immobilization device, in accordance with an embodiment of the present invention. In some embodiments, the immobilization device may include a portable, single-use device that may be robotically deployed to protect patients who may suffer from a spine or other serious injury requiring immobilization for safe movement. The immobilization device may employ an inflatable system that conforms to the patient's body contour and creates a comfortable stretcher for the patient, with a rugged, durable structure that maintains its integrity through robotic lifting and casualty transportation. Those skilled in the art will readily recognize, in light of and in accordance with the teachings of the present invention, that the immobilization device may greatly reduce the potential for further damage to the spine during lifting and transport of the patient, while providing easy access to the patient's chest or abdominal area for treatment of diagnostic procedures. In one alternative embodiment, the immobilization device may be configured to stabilize numerous body parts, including, without limitation, the arm, the wrist, the leg, and the torso.

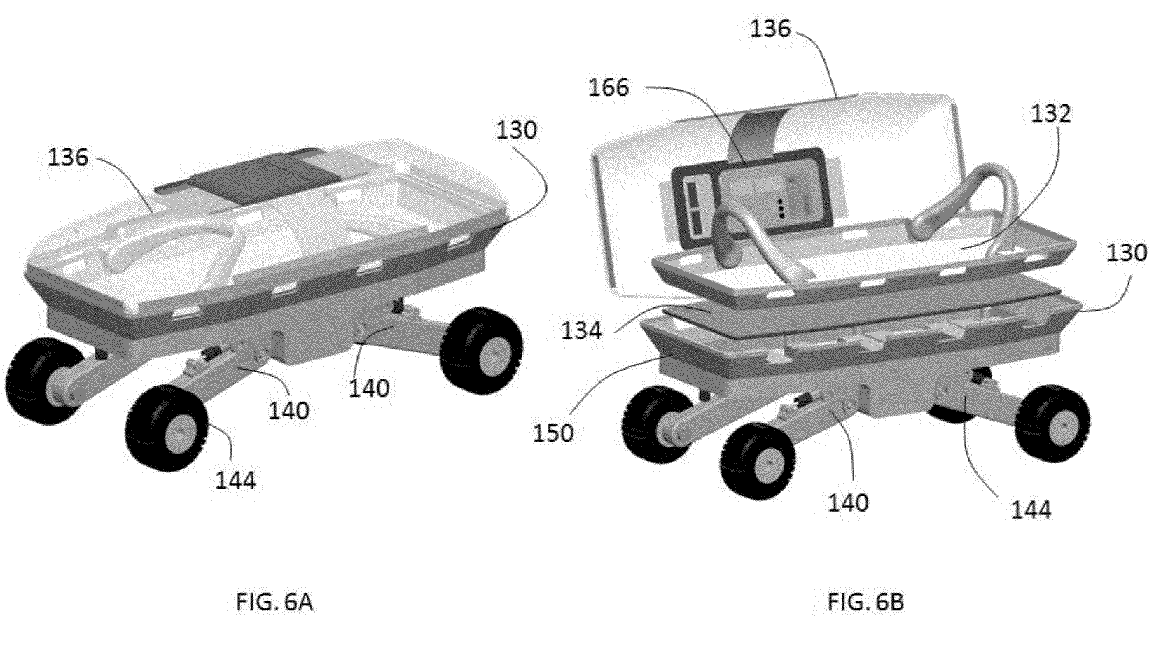

FIGS. 6A and 6B illustrate detailed perspective views of an exemplary extraction vehicle 130, in accordance with an embodiment of the present invention, where FIG. 6A illustrates an exemplary extraction vehicle with the lid closed, and FIG. 6B illustrates an exemplary extraction vehicle with the lid open. In some embodiments, the extraction vehicle may include a secure, bullet-proof wheeled system that may be pulled by the robot first responder to safety, and then may continue to serve as the evacuation stretcher to a medical facility. In some embodiments, the extraction vehicle may be motorized and include actuations for mobility mechanism, navigation sensors, and continuous patient monitoring 166. In some embodiments, the extraction vehicle may also include data capture capacities for bringing instant wireless vital signs communication between each extraction vehicle and the remote operator, either directly, or through the robot first responder. The extraction vehicle may utilize a wireless sensor/radio transceiver network to track data internal to each extraction vehicle, transmit data between different extraction vehicles, and transmit information to external entities as required. In some embodiments, the patient may be inconstant communication with the remote operator. In one alternative embodiment, the extraction vehicle may include a thermometer, a heart monitoring device, and a video camera to remotely monitor the patient's situation while being transported.

In some embodiments, the extraction vehicle may utilize a passive mobility mechanism that links with the robot first responder so that it can be pulled from the combat zone to safety and subsequent evacuation. In some embodiments, the extraction vehicle may operate be remote control from a remote operator. In some embodiments, the extraction vehicle may incorporate power drive and autonomous navigation systems, enabling multiple extraction vehicles to self-navigate to safety after receiving the injured patient. Suitable materials for the extraction vehicle may include, without limitation, titanium, steel, iron, rubber, plastic, and metal alloys. In some embodiments, the extraction vehicle may include a portable power source. However, in other embodiments, the robot first responder may tow the extraction vehicle.

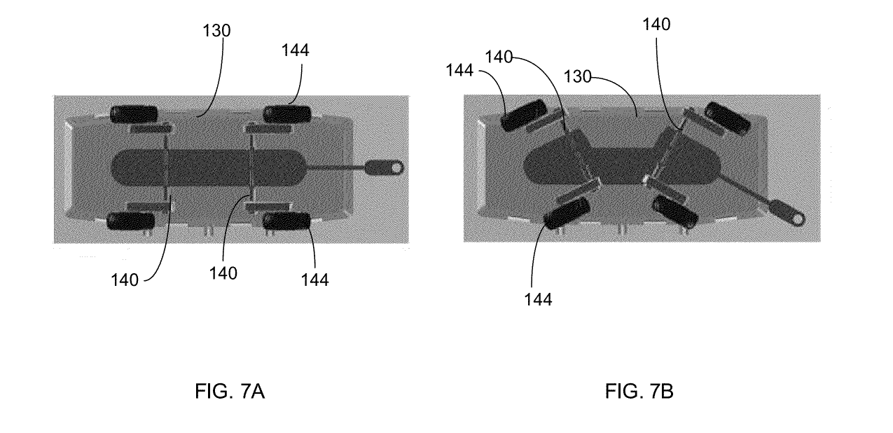

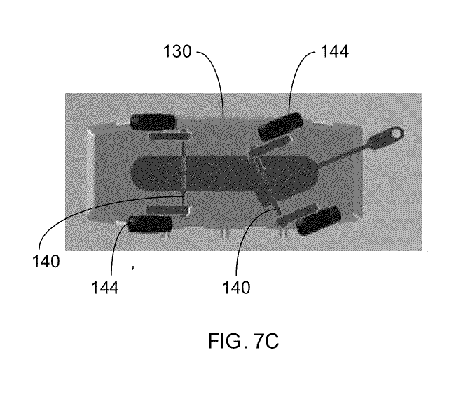

FIGS. 7A, 7B, and 7C illustrate bottom views of an exemplary axle assembly 140 in various positions, in accordance with an embodiment of the present invention. FIGS. 7A and 7B illustrate the front axle assembly and the rear axle assembly steering independently and under motor control. In some embodiments, the independent steering may be possible because the axle assembly of each pair of wheels intersects the vertical pivot center of the steering pivot. FIG. 7C illustrates the rear axle assembly locked in a front-back orientation for towing mode. When in the towing mode, the rear axle assembly may be locked in a front-back orientation, and the front axle assembly may be allowed to freely respond to the pulling action of a detachable tow bar used by the robot first responder.

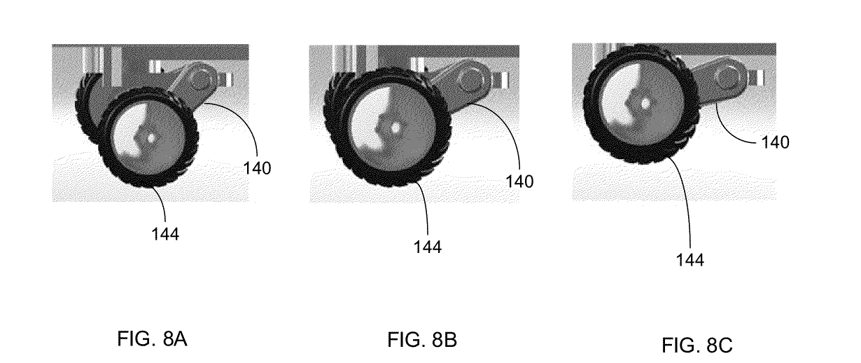

FIGS. 8A, 8B, and 8C illustrate detailed perspective views of an exemplary sequential image of a pair of wheels 144 with a unique 4-wheel methodology, providing simultaneous support for bump absorption and heading control wheel on the axle assembly in three varying positions, in accordance with an embodiment of the present invention. In some embodiments, each pair of wheels (2×front, 2×back) may be independently steered under motor control. Those skilled in the art will readily recognize, in light of and in accordance with the teachings of the present invention, that since the axle assembly of each pair of wheels intersects the vertical pivot center of the steering pivot, each wheel does not drag, thereby providing easy low-torque steering. Suitable materials for the wheels may include, without limitation, rubber, plastic, metal alloys, and silicone.

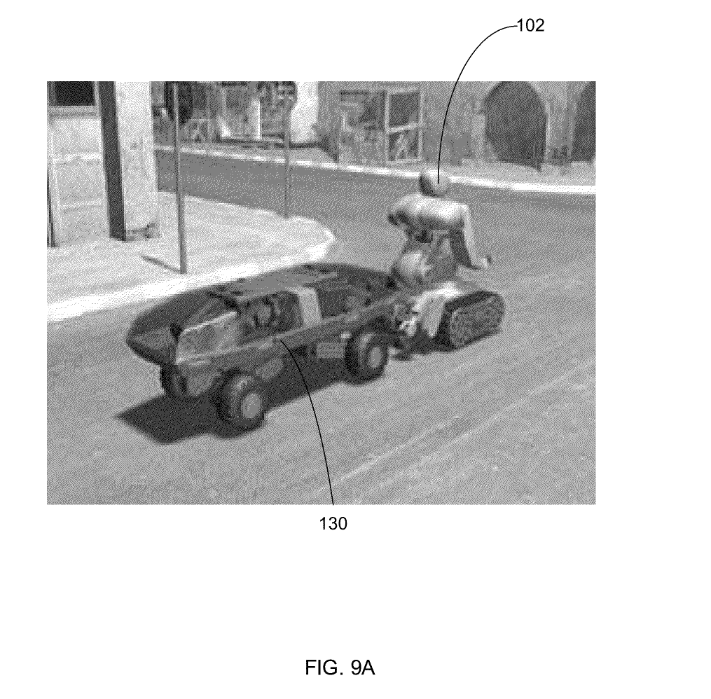

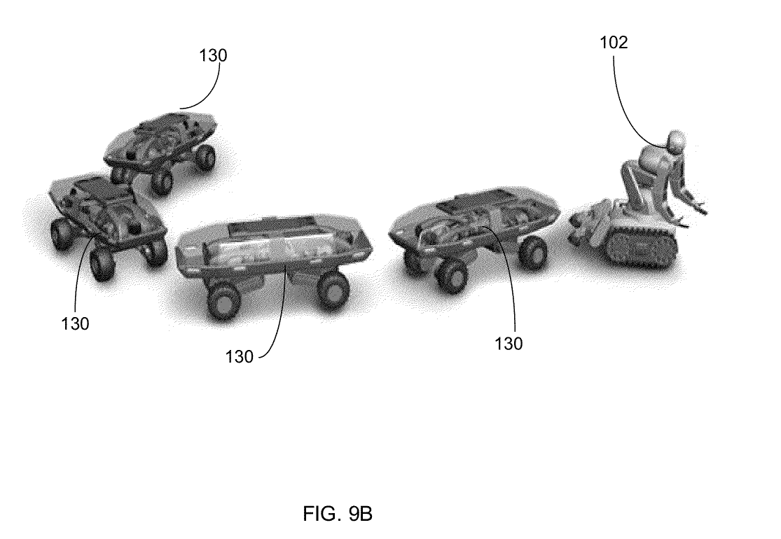

FIGS. 9A and 9B illustrate detailed perspective views of an exemplary robot first responder towing the extraction vehicle 130, in accordance with an embodiment of the present invention, where FIG. 9A illustrates an exemplary scenario of the extraction vehicle being towed by the robot first responder, and FIG. 9B illustrates an exemplary scenario of the extraction vehicle incorporating power drive and autonomous navigation systems, which enable multiple extraction vehicles to self-navigate to safety after receiving the injured patient. Those skilled in the art, in light of the present teachings, will recognize that the concept design of a motorized extraction vehicle may utilize a network of extraction vehicle systems communicating external commands through ad-hoc networking protocol.

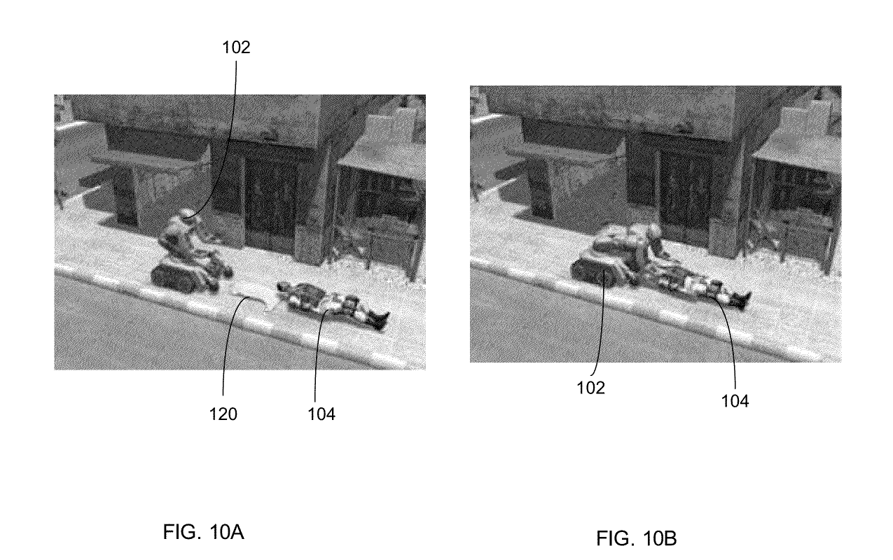

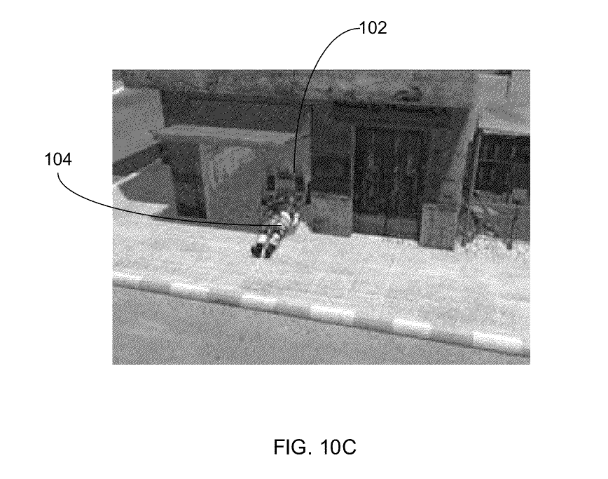

FIGS. 10A, 10B, and 10C illustrate detailed perspective views of an exemplary scenario of the robot first responder pulling the patient to safety in a combat zone, in accordance with an embodiment of the present invention. Those skilled in the art will readily recognize, in light of and in accordance with the teachings of the present invention, that the robotic first response systems and methods may be efficacious in rescuing soldiers who have been injured in a battlefield scenario. FIG. 10A illustrates an exemplary robot first responder approaching and assessing the soldier. In some embodiments, the robot first responder may be bullet proof and impervious to debris.

FIG. 10B illustrates an exemplary robot first responder preparing the soldier to be extracted from the combat zone. In some embodiments, the robot first responder may position the soldier on the stabilizing device and inflate the stabilizing device to conform to the soldier's contours. FIG. 10C illustrates the robot first responder pulling the soldier to a secure location. Those skilled in the art will readily recognize, in light of and in accordance with the teachings of the present invention, that the robot first responder may not always be able to extract the soldier directly to the extraction vehicle if the situation is dangerous or there is propelled debris in the vicinity. The remote operator may utilize supervisory confirmation between each phase of the operation to make ad-hock decisions in an evolving battlefield scenario.

In some alternative embodiments, the robot first responder may utilize speech recognition software to identify the injured soldier, or move towards a particular soldier in distress. The robot first responder may also speak to the soldier in a soothing human voice to alleviate trepidation from soldier. In yet another alternative embodiment, dexterous manipulators may perform surgery on the soldier in the combat zone through direction from the remote operator.

It should be understood that while the foregoing embodiments were described in terms of a soldier, alternative embodiments would similarly apply, without limitation, generally to medial patients, disaster victims, and conflict casualties. Similarly, while the foregoing embodiments were described in terms delivering or enabling medical care to be provided, alternative embodiments of the present invention may be directed towards aiding in the recovery, disposal, or otherwise robotically handling dead persons, depending upon the particular needs of the application.

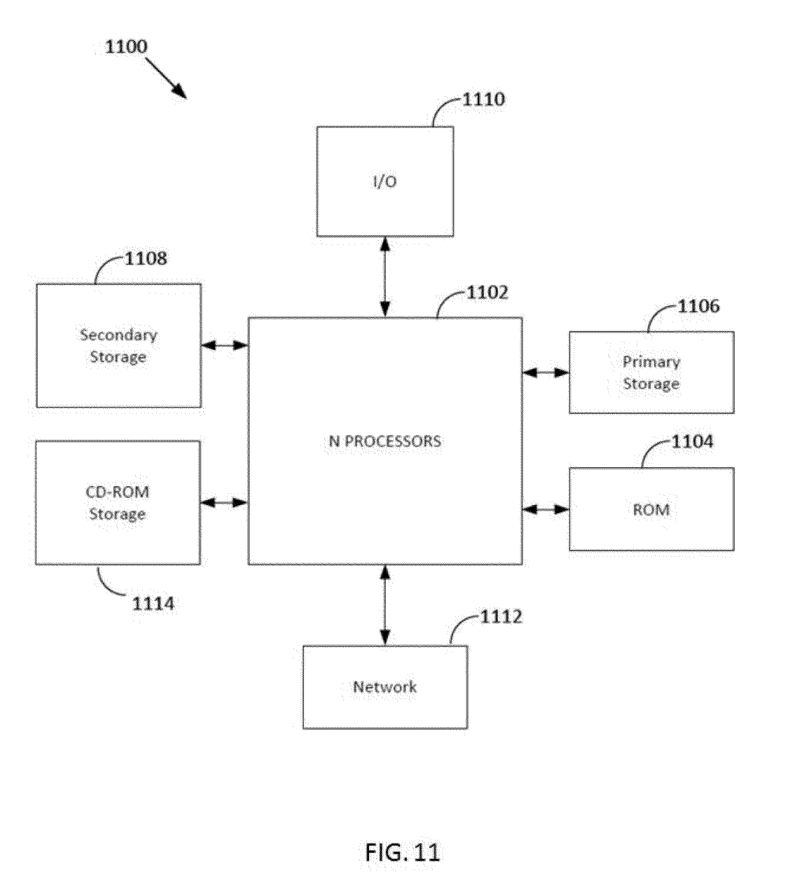

FIG. 11 illustrates a typical computer system that, when appropriately configured or designed, can serve as a computer system 1100 in which the present invention may be embodied. Computer system 1100 includes a quantity of processors 1102 (also referred to as central processing units, or CPUs) that are coupled to storage devices including a primary storage 1106 (typically a random access memory, or RAM), a primary storage 1104 (typically a read only memory, or ROM). CPU 1102 may be of various types including micro-controllers (e.g., with embedded RAM/ROM) and microprocessors such as programmable devices (e.g., RISC or SISC based, or CPLDs and FPGAs) and devices not capable of being programmed such as gate array ASICs (Application Specific Integrated Circuits) or general purpose microprocessors. As is well known in the art, primary storage 1104 acts to transfer data and instructions uni-directionally to the CPU and primary storage 1106 is used typically to transfer data and instructions in a bi-directional manner. The primary storage devices discussed previously may include any suitable computer-readable media such as those described above. A mass storage device 1108 may also be coupled bi-directionally to CPU 1102 and provides additional data storage capacity and may include any of the computer-readable media described above. Mass storage device 1108 may be used to store programs, data and the like and is typically a secondary storage medium such as a hard disk. It will be appreciated that the information retained within the mass storage device 1108, may, in appropriate cases, be incorporated in standard fashion as part of primary storage 1106 as virtual memory. A specific mass storage device such as a CD-ROM 1114 may also pass data uni-directionally to the CPU.

CPU 1102 may also be coupled to an interface 1110 that connects to one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 1102 optionally may be coupled to an external device such as a database or a computer or telecommunications or internet network using an external connection shown generally as a network 1112, which may be implemented as a hardwired or wireless communications link using suitable conventional technologies. With such a connection, the CPU might receive information from the network, or might output information to the network in the course of performing the method steps described in the teachings of the present invention.

Those skilled in the art will readily recognize, in light of and in accordance with the teachings of the present invention, that any of the foregoing steps and/or system modules may be suitably replaced, reordered, removed and additional steps and/or system modules may be inserted depending upon the needs of the particular application, and that the systems of the foregoing embodiments may be implemented using any of a wide variety of suitable processes and system modules, and is not limited to any particular computer hardware, software, middleware, firmware, microcode and the like. For any method steps described in the present application that can be carried out on a computing machine, a typical computer system can, when appropriately configured or designed, serve as a computer system in which those aspects of the invention may be embodied.

All the features or embodiment components disclosed in this specification, including any accompanying abstract and drawings, unless expressly stated otherwise, may be replaced by alternative features or components serving the same, equivalent or similar purpose as known by those skilled in the art to achieve the same, equivalent, suitable, or similar results by such alternative feature(s) or component(s) providing a similar function by virtue of their having known suitable properties for the intended purpose. Thus, unless expressly stated otherwise, each feature disclosed is one example only of a generic series of equivalent, or suitable, or similar features known or knowable to those skilled in the art without requiring undue experimentation.

Having fully described at least one embodiment of the present invention, other equivalent or alternative methods of implementing extraction of an injured soldier in a combat through a robot first responder, immobilization device, and extraction vehicle according to the present invention will be apparent to those skilled in the art. Various aspects of the invention have been described above by way of illustration, and the specific embodiments disclosed are not intended to limit the invention to the particular forms disclosed. The particular implementation of the extraction of an injured soldier in a combat through a robot first responder, stabilizing device, and extraction vehicle may vary depending upon the particular context or application. By way of example, and not limitation, the robotic first responder systems and methods described in the foregoing were principally directed to extraction of an injured soldier in a combat through a robot first responder, stabilizing device, and extraction vehicle; however, similar techniques may instead be applied to a forest fire first responder, whereby the robot first responder is fire proof and ejects fire retardants, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

What is claimed is:

1. A robotic first responder system comprising:
a robotic first responder, said robotic first responder being operable to provide medical care to a patient, or a combat casualty, or a disaster casualty;
an immobilization device, said immobilization device being configured to receive and support said patient; and
an extraction vehicle, said extraction vehicle being operable to transport said patient.

2. The robotic first responder system of claim 1, wherein said robotic first responder is operable to provide first responder medical care to said patient or casualty.

3. The robotic first responder system of claim 2, in which said robotic first responder comprises a humanoid upper torso.

4. The robotic first responder system of claim 3, in which said robotic first responder comprises at least one bimanual dexterous manipulator, said at least one bimanual dexterous manipulator being configured to manipulate said patient or casualty.

5. The robotic first responder system of claim 4, in which said robotic first responder comprises a stability enhancement device, said stability enhancement device being operable to provide stability to said robotic first responder when lifting and transporting said patient or casualty.

6. The robotic first responder system of claim 5, wherein said robotic first responder is configured to be bullet-proof.

7. The robotic first responder system of claim 6, wherein said robotic first responder tows said extraction vehicle.

8. The robotic first responder system of claim 7, wherein said immobilization device is operable to inflate with fast-hardening foam material by said robotic first responder.

9. The robotic first responder system of claim 8, wherein said immobilization device is configured to contour said patient's neck and spine.

10. The robotic first responder system of claim 9, in which said extraction vehicle comprises a base platform, said base platform being configured to receive said patient or casualty.

11. The robotic first responder system of claim 10, in which said extraction vehicle comprises a lifting device, said lifting device being configured to elevate said patient or casualty onto said extraction vehicle.

12. The robotic first responder system of claim 11, in which said extraction vehicle comprises a lid portion, said lid portion being configured to cover said patient or casualty inside said extraction vehicle.

13. The robotic first responder system of claim 12, in which said extraction vehicle comprises a navigation sensory portion, said navigation sensory portion being operable to guide said extraction vehicle.

14. The robotic first responder system of claim 13, in which said extraction vehicle comprises a vital signs recording device, said vital signs recording device being operable to monitor and record vital signs of said patient or casualty.

15. The robotic first responder system of claim 14, in which said extraction vehicle comprises a transmitter/receiver device, said transmitter/receiver device being operable to provide two-way communications.

16. The robotic first responder system of claim 15, in which said extraction vehicle comprises a pair of wheels or tracks.

17. The robotic first responder system of claim 16, in which said pair of wheels comprises a unique 4-wheel methodology, said 4-wheel methodology being operable to provide simultaneous support for bump absorption and a heading control wheel on an axle assembly in varying positions.

18. The robotic first responder system of claim 17, wherein said extraction vehicle is configured to be bullet-proof.

19. A robotic first responder method comprising:
means for assessing a patient or casualty;
means for receiving supervisory confirmation;
means for preparing said patient or casualty for transport;
means for positioning said patient or casualty on an immobilization device;
means for receiving supervisory confirmation;
means for lifting said patient or casualty;
means for carrying said patient or casualty to an extraction vehicle; and
means for transporting said patient or casualty.

20. A robotic first responder system comprising:
a robotic first responder, said robotic first responder being operable to provide medical care to a patient, said robotic first responder comprising a humanoid upper torso, said robotic first responder further comprising at least one bimanual dexterous manipulator, said robotic first responder further comprising a stability enhancement device, said robotic first responder further comprising a navigation control system, said robotic first responder comprising at least four compliant actuators in each bimanual dexterous manipulators;
an immobilization device, said immobilization device being configured to receive and support said patient, said immobilization device being operable to inflate with air; and
an extraction vehicle, said extraction vehicle being operable to transport said patient, said extraction vehicle comprising a base platform, said extraction vehicle further comprising a lifting device, said extraction vehicle further comprising a lid portion, said extraction vehicle further comprising a pair of wheels, said extraction vehicle further comprising a navigation sensory portion.
